(19) United States
(12) Patent Application Publication
(10) Pub. No.: US 2025/0325392 A1
(43) Pub. Date: Oct. 23, 2025

(54) STENT FOR TREATMENT OF TINNITUS

(71) Applicant: Sonorous NV, Lake Forest, CA (US)

(72) Inventors: Dave Ferrera, Coto De Caza, CA (US); Jake Le, Lake Forest, CA (US)

(21) Appl. No.: 18/641,197

(22) Filed: Apr. 19, 2024

(51) Int. Cl. A61F 2/966; A61F 2/06; A61F 2/95; A61M 29/02

(52) U.S. Cl. CPC A61F 2/966; A61M 29/02; A61F 2002/068; A61F 2002/9534; A61F 2250/0039; A61F 2250/0098; A61M 2029/025

(57) ABSTRACT

A stent device includes a single wire forming a braided body that extends between a proximal end and a distal end. The wire is formed of drawn filled tube (DFT). The braided body has a braid density in a range of 15 programmable picks per inch (PPI) to 34 PPI. The stent device is configured for implantation in a cerebral dural venous sinus and, when implanted, is configured to generate a chronic outward force sufficient to change blood flow within the cerebral dural venous sinus from pulsatile flow or turbulent flow to laminar flow.

### TECHNICAL FIELD

This disclosure relates to stents and related delivery systems, in particular stent devices and systems to treat pulsatile tinnitus.

### BACKGROUND

Cerebral venous sinus stenosis is a disease that obstructs venous blood outflow in the cerebral dural venous sinuses. Cerebral venous sinus stenosis can be caused by swollen brain matter applying pressure to the sinuses (extrinsic stenosis), or internal stenosis caused by protrusions external to the sinuses (e.g., arachnoid granulations) pressing on the sinuses, diverticula, or dehiscence. Cerebral venous sinus stenosis may cause increased intravenous pressure and reduced regional blood flow, thus resulting in headaches, cognitive impairment, and progressive visual loss. If left untreated, cerebral venous sinus stenosis (CVSS) can cause optic nerve damage and permanent vision loss.

In addition, CVSS can result in pulsatile tinnitus, which causes a person to hear rhythmic thumping, whooshing, or throbbing synchronous to their heartbeat in one or both ears and can result in significantly reduced quality of life. Other abnormalities or disorders that can cause pulsatile tinnitus include extrinsic stenosis, arachnoid granulations, diverticulum, dehiscence, and high riding jugular bulb.

### SUMMARY

In an example implementation, a stent device includes a single wire forming a braided body that extends between a proximal end and a distal end. The wire is formed of drawn filled tube (DFT). The braided body has a braid density in a range of 15 programmable picks per inch (PPI) to 34 PPI. The stent device is configured for implantation in a cerebral dural venous sinus and, when implanted, is configured to generate a chronic outward force sufficient to change blood flow within the cerebral dural venous sinus from pulsatile flow or turbulent flow to laminar flow.

Embodiments can include one or more of the following features in any combination.

In certain embodiments, the stent device further includes a flared crown on at least one of the proximal end and the distal end.

In some embodiments, the chronic outward force generated by the stent device is at least 0.036 N/mm.

In certain embodiments, the chronic outward force generated by the stent device is in a range of at 0.020 N/mm and 0.050 N/mm.

In some embodiments, the chronic outward force generated by the stent device is in a range of at 0.030 N/mm and 0.040 N/mm.

In certain embodiments, the chronic outward force generated by the stent device is in a range of at 0.030 N/mm and 0.035 N/mm.

In some embodiments, the chronic outward force generated by the stent device is in a range of at 0.0197 N/mm and 0.0433 N/mm.

In certain embodiments, an unconstrained length of the braided body is 45 mm.

In some embodiments, a diameter of the wire is between 0.0035 inches and 0.0065 inches.

In certain embodiments, the braid density is in a range of 23 PPI to 34 PPI.

In some embodiments, an unconstrained length of the braided body is 60 mm.

In certain embodiments, a diameter of the wire is between 0.0035 inches and 0.0065 inches.

In some embodiments, the braid density is in a range of 15 PPI to 21 PPI.

In certain embodiments, the stent device is configured to be inserted into the cerebral dural venous sinus using a 5 French catheter. the stent device is configured to be inserted into the cerebral dural venous sinus using a 5 French catheter.

In some embodiments, the stent device further includes at least one coil of DFT wire coiled about the braided body at the proximal end, and the at least one coil is configured to releasably couple the stent device to an insertion device.

In certain embodiments, a resistive radial force generated by the stent device is in a range of 0.170 N/mm and 0.700 N/mm.

In some embodiments, a resistive radial force generated by the stent device is in a range of 0.300 N/mm and 0.500 N/mm.

In certain embodiments, a resistive radial force generated by the stent device is in a range of 0.300 N/mm and 0.400 N/mm.

In some embodiments, a resistive radial force generated by the stent device is in a range of 0.1651 N/mm and 0.2998 N/mm.

In certain embodiments, a resistive radial force generated by the stent device is in a range of 0.20299 N/mm and 0.54337 N/mm.

In another aspect, a system includes a stent device and an insertion device. The stent device includes a single wire forming a braided body that extends between a proximal end and a distal end. The stent device is configured for implantation in a cerebral dural venous sinus and, when implanted, is configured to generate a chronic outward force sufficient to change blood flow within the cerebral dural venous sinus from pulsatile flow or turbulent flow to laminar flow. The insertion device includes a pusher rod, a stent body coil positioned proximate an end of the pusher rod, a radiopaque pusher body coil coupled to a portion of the pusher rod, a polymer tube coupled to the pusher rod between the stent body coil and the pusher body coil, and a catheter configured to enclose the stent device and the pusher rod. The stent body coil is configured to extend through at least a portion of the stent device when the stent device is coupled to the insertion device.

Embodiments can include one or more of the following features in any combination.

In certain embodiments, the stent device further includes at least one coil of DFT wire coiled about the braided body at the proximal end, wherein the at least one coil is configured to releasably couple the stent device to an insertion device; and the stent device is releasably coupled to the insertion device by friction between the at least one coil, the polymer tube, and an inner surface of the catheter

In some embodiments, when the stent device is coupled to the insertion device, the at least one coil is positioned proximal to the polymer tube.

In certain embodiments, the at least one coil includes two coils positioned on opposite loops of the proximal end of the braid body.

In some embodiments, the single wire is formed of DFT.

In certain embodiments, the braided body has a braid density in a range of 15 programmable picks per inch (PPI) to 34 PPI.

In some embodiments, the stent device is resheathable into the catheter when approximately 90% or less of the braid body has been deployed outside the catheter.

In certain embodiments, the catheter is a 5 French catheter.

In some embodiments, the pusher body coil is configured to prevent kinking of the pusher rod during insertion of the stent device in the cerebral dural venous sinus.

In another aspect, a method includes inserting a catheter into a target vessel of dural venous sinuses of a patient; inserting an insertion device and a stent device releasably coupled to the insertion device through the catheter; positioning a distal end of the stent device proximate a distal end of the catheter; and withdrawing the catheter proximally to release the stent device from the insertion device and implant the insertion device into the target vessel, wherein when implanted, the stent device is configured to generate a chronic outward force sufficient to change blood flow within the target vessel from pulsatile flow or turbulent flow to laminar flow.

Embodiments can include one or more of the following features in any combination.

In certain embodiments, the stent device includes a single wire forming a braided body that extends between a proximal end and a distal end.

In some embodiments, the stent device further includes at least one coil of DFT wire coiled about the braided body at the proximal end; the insertion device includes a pusher rod and a polymer tube positioned over the pusher rod; and when the stent device is coupled to the insertion device, the at least one coil is positioned proximal to the polymer tube.

In certain embodiments, releasing the stent device from the insertion device includes withdrawing the catheter proximally until the polymer tube is deployed outside a distal end of the catheter.

In some embodiments, the catheter is a 5 French catheter

In certain embodiments, the method further includes prior to releasing the stent device from the insertion device: resheathing the stent device within the catheter and repositioning the catheter within the target vessel.

In some embodiments, resheathing of the stent device into the catheter is performed when approximately 90% or less of the stent device has been deployed outside the catheter.

In some embodiments, the method further includes withdrawing the insertion device proximally out of the catheter and withdrawing the catheter out of the cerebral dural venous sinuses of the patient.

In certain embodiments, implantation of the stent device in the target vessel is performed using medical imaging.

Advantages of the systems, devices, and methods described herein can include restoration of laminar flow within the cerebral dural venous sinus, which can result in reduction or elimination of pulsatile tinnitus. The system, devices, and methods described herein can also allow a stent device to be visually monitored using medical imaging as it is placed in the cerebral dural venous sinus, making placement of the stent device safer, faster, and more accurate. The system, devices, and methods described herein enable resheathing or recapturing of the stent device before implantation of the stent device in a target blood vessel, which can result in improved placement of the stent device within the cerebral dural venous sinus by enabling the clinician to reposition the stent device following partial deployment of the stent device. The system, devices, and methods described herein enable a stent device to be deployed within the cerebral dural venous sinus using a 5 French (F) or smaller catheter, which allows for easy and fast placement of the stent device in the cerebral dural venous sinus. The system, devices, and methods described herein enable the stent device to remain patent and reduce the risk of the stent device kinking when the stent device traverses a curve, such as when the stent device curves around the anatomy of the cerebral dural venous sinus of a patient. The systems, devices, and methods described herein enable stenting of both the transverse and sigmoid sinuses to prevent adjacent stenosis.

The details of one or more embodiments of the disclosure are set forth in the accompanying drawings and the description below. Other features, objects, and advantages will be apparent from the description and drawings, and from the claims.

### DESCRIPTION OF DRAWINGS

FIG. 1 depicts a perspective view of a stent device for treatment of tinnitus.

FIG. 2 depicts a side view of the stent device of FIG. 1.

FIG. 3 depicts an end view of the stent device of FIG. 1.

FIG. 4A depicts implantation of the stent of FIG. 1 in the transverse sinus of a patient.

FIG. 4B depicts a cross section view of the transverse sinus along axis A-A depicted in FIG. 4A.

FIG. 4C depicts a cross section view of the transverse sinus along axis B-B depicted in FIG. 4A.

FIG. 4D depicts a cross section view of the transverse sinus along axis B-B depicted in FIG. 4A following implantation of the stent device of FIG. 1.

FIG. 5 depicts an example insertion device for implanting the stent device of FIG. 1.

FIG. 6 depicts the stent device of FIG. 1 and the insertion device of FIG. 5 loaded into a catheter for implantation of the stent device.

FIG. 7 depicts a connection of the stent device of FIG. 1 to the insertion device of FIG. 5.

FIG. 8A-8C depict end views of example stent devices.

FIG. 9 depicts example medical imaging of the stent device of FIG. 1 being implanted within a target vessel.

FIG. 10 depicts an example method of implanting the stent device of FIG. 1.

FIG. 11 depicts a table summarizing patient characteristics of patients treated with the stent device of FIG. 1 during a clinical study.

FIG. 12 depicts a table summarizing treatment outcomes for patients treated with the stent device of FIG. 1 during a clinical study.

FIG. 13 depicts tables summarizing the resistive radial force measured for example stent devices.

Like reference symbols in the various drawings indicate like elements.

### DETAILED DESCRIPTION

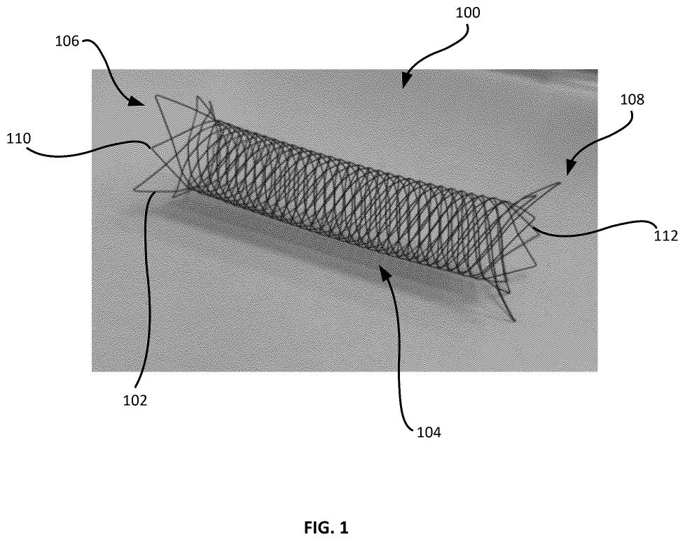

FIG. 1 depicts a stent device 100 for treatment of pulsatile tinnitus. As depicted in FIGS. 1-3, the stent device 100 includes a single wire 102 that is wound to form a braided stent body 104. The braided stent body 104 extends between a proximal end 106 of the stent device 100 and a distal end 108 of the stent device 100.

The length 124 of the stent device 100 prior to implantation (“unconstrained length”) can be in a range between 30 mm and 100 mm. In some implementations, the unconstrained length 124 of the stent device 100 is 33 mm. In some implementations, the unconstrained length 124 of the stent device 100 is 40 mm. In some implementations, the unconstrained length 124 of the stent device 100 is 45 mm. In some implementations, the unconstrained length 124 of the stent device 100 is 60 mm. In some implementations, unconstrained length 124 of the stent device 100 is selected based on the anatomy of the patient. For example, the unconstrained length 124 of the stent device 100 can be selected based on the distance from the torcula of a patient through the transverse sinus and the sigmoid sinus of the patient such that the stent device 100 can be deployed within these vessels of the patient while minimizing the risk of stenosis development following placement of the stent device 100.

The unconstrained diameter 126 of the stent body 104 prior to implantation (“unconstrained diameter”) can be in a range between 6 mm and 10 mm. In some implementations, the unconstrained diameter 126 of the stent body 104 is 8 mm. In some implementations, the unconstrained diameter 126 of the stent body 104 is 10 mm. In some implementations, the unconstrained diameter 126 of the stent body 104 is selected based on the anatomy of the patient. For example, the unconstrained diameter 126 of the stent body 104 selected for a patient can be determined based on the diameter of the blood vessels of the patient's dural venous sinuses.

In some implementations, the unconstrained length 124 of the stent device 100 is 45 mm and the unconstrained diameter 126 of the stent body 104 is 8 mm. In some implementations, the unconstrained length 124 of the stent device 100 is 60 mm and the unconstrained diameter 126 of the stent body 104 is 8 mm. In some implementations, the unconstrained length 124 of the stent device 100 is 33 mm and the unconstrained diameter 126 of the stent body 104 is 10 mm. In some implementations, the unconstrained length 124 of the stent device 100 is 40 mm and the unconstrained diameter 126 of the stent body 104 is 10 mm.

In some implementations, the wire 102 used to form the stent device is a drawn filled tube (DFT) wire. The wire 102 can be radiopaque in order to enable visualization of the stent device 100 using medical imaging (e.g., digital subtraction angiography or any other suitable medical imaging technique). In some implementation, the wire 102 is a DFT wire that is formed of a micro-hypotube that is formed of a first material selected for elasticity and is filled with a second, radiopaque material. In some implementations, the wire 102 is a DFT wire that is formed of a nickel-titanium alloy (e.g., nitinol) tube that is filled with platinum. The nickel-titanium of the DFT wire 102 allow provides the stent device 100 with sufficient elasticity to navigate the bends and tortuous paths of the cerebral dural venous sinuses of the patient without kinking or losing patency. The platinum of the DFT wire 102 causes the stent device 100 to be radiopaque such that a clinician can visualize the stent device 100 throughout the placement procedure using medical imaging.

In some implementations, the wire 102 used to form the stent device 100 is formed of a material that includes a shape-memory alloy, such as nitinol (NiTi). As a result, the stent device 100 can be formed by braiding the wire 102 to form the stent body 104, and then heat treating the stent body 104 to set and maintain the shape of the braided stent device 100. As a result, the stent device 100 can be compressed into a catheter for navigating the stent device 100 to a target blood vessel and self-expands to the desired shape once implanted in the target blood vessel.

In some implementations, the diameter of the wire 102 used to form the stent body 104 is in a range of 0.0035 inches and 0.0065 inches. In some implementations, the unconstrained length 124 of the stent device 100 is 45 mm and the diameter of the wire 102 is in a range 0.0035 inches and 0.0065 inches. In some implementations, the unconstrained length 124 of the stent device 100 is 60 mm and the diameter of the wire 102 is in a range 0.0035 inches and 0.0065 inches. In some implementations, the diameter of the wire 102 is 0.0055 inches. In some implementations, the wire 102 is electropolished and passivated after winding the wire 102 to form the braided stent body 104. As will be described in further detail herein, the diameter of the wire 102 used to form the stent device 100 influences several mechanical properties of the stent device 100.

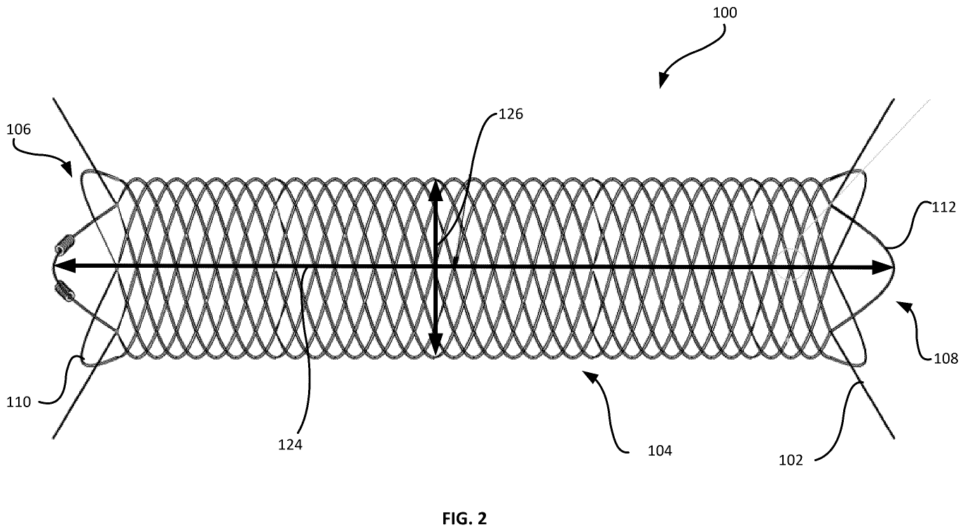

As can be seen in FIGS. 1 and 2, a single wire 102 is wound multiple times in order to form the braided stent body 104. In some implementations, the braided stent body 104 is formed by winding the wire 102 around a series of pins on a mandrel according to a specific winding pattern. Once the winding of the wire 102 is complete, the opposite ends of the wire 102 can be welded together to form the stent body 104. As a result of the single wire braided design of the stent device, the stent device 100 is a “closed end” stent that does not include any exposed ends of wire, which reduces the risk of damage or stress to the blood vessel in which the stent device 100 is implanted. In addition, because the stent device 100 is formed by braiding a single wire, the ends of the stent device 100 do not require soldering, which increase durability of the stent device 100 and reduces risk of injury to the blood vessel. In addition, as will be discussed in further detail herein, the single wire, closed cell, braided design of the stent device 100 enables the stent device 100 to be resheathed for repositioning or removal of the stent 100 during the implantation process following partial deployment of the stent device 100.

The braided design of the stent device 100 also helps the stent 100 to remain open (patent) and resist kinking when implanted across curved or tortuous paths. For example, as previously discussed, the stent device 100 is formed by winding a single wire 102 about a mandrel and the two ends of the wire 102 being welded together. As a result, the intersections (“picks”) of the wire 102 in the braided stent body 104 are not welded together or otherwise connected to one another, which enables crisscrossing portions of the wire 102 to slide past one another when the stent device 100 subjected to curves or other forms of compression. Due to this structure of movable picks along the braided stent body 104, the stent device 100 can remain patent and resist kinking when positioned along curving pathways. In some implementations, the stent device 100 remains at least 95% patent when traversing a 90-degree curve.

The pitch and the number of windings of the wire 102 in the stent body 104 can be adjusted during manufacturing to provide a particular braid density. In some implementations, the braid density of the stent device 100 is in a range of 15 programmable picks per inch (PPI) to 34 PPI. The braid density of the stent device 100 is directly correlated to the radio density of the stent device 100, and the braid density of the stent device 100 can be selected to optimize radiopacity of the stent device 100 in order to enable visualization of the stent device 100 during implantation.

In some implementations, the braid density of the stent device 100 is selected based on the unconstrained length 124 of the stent device 100. For example, in some implementations, the unconstrained length 124 of the stent device 100 is 45 mm and the braid density of the stent device 100 is in a range of 23 PPI to 34 PPI. In some implementations, the unconstrained length 124 of the stent device 100 is 60 mm and the braid density of the stent device 100 is in a range of 15 PPI to 21 PPI.

Once the stent device 100 has been braided using the techniques described above, the stent device 100 is heat treated in order to set the shape, including the diameter, of the stent device 100. The austenite start and finish temperatures and the martensite start and finish temperatures with which the stent device 100 is heat treated can be selected in order to cause the stent device 100 to have shape memory that maintains the braid pattern and density of the stent device 100, as well as the diameter of the stent body 104 and the diameter of the flared crowns 110, 112. As a result, the stent device 100 can be compressed (e.g., inside a catheter) during delivery of the device to the target blood vessel and can self-expands into the desired shape and contacts the blood vessel walls once implanted in the target blood vessel. As will be discussed in further detail herein, the braid density and diameter of the stent device 100 influence the chronic outward force and resistive radial force exhibited by the stent device 100. Therefore, self-expansion of the stent device into the desired shape is an important feature of the stent device 100.

The diameter of the wire 102 that is used to form the stent device 100, the diameter of the stent body 104, and the braid density of stent device 100 each influence several mechanical properties of the stent device 100, including the elasticity of the stent device 100, the chronic outward force produced by the stent device 100, and the resistive radial force of the stent device 100. For example, increasing the diameter of the wire 102, increasing the braid density of the stent device 100, or both, increases the chronic outward force produced by the stent device 100, increases the resistive radial force of the stent device 100, and reduces the elasticity of the stent device 100. Thus, the combination of the diameter of the wire 102 used to form the stent device 100 and the braid density of stent device 100 can be selected to provide the desired mechanical properties for the stent device 100.

The chronic outward force of the stent device 100 corresponds to the force that the stent device 100 continuously applies to a blood vessel following implantation of the stent device 100 within the blood vessel. In some implementations, the chronic outward force produced by the stent device 100 following implantation is at least 0.036 N/mm. In some implementations, the chronic outward force produced by the stent device 100 following implantation is in a range of 0.020 N/mm and 0.050 N/mm. In some implementations, the chronic outward force produced by the stent device 100 following implantation is in a range of 0.030 N/mm and 0.040 N/mm. In some implementations, the chronic outward force produced by the stent device 100 following implantation is in a range of 0.030 N/mm and 0.035 N/mm. In some implementations, the chronic outward force produced by the stent device 100 following implantation is in a range of 0.0197 N/mm and 0.0433 N/mm. In some implementations, the chronic outward force generated by the stent device 100 is sufficient to change the blood flow within the blood vessel in which the stent device 100 is implanted from pulsatile flow or turbulent flow to laminar flow. As a result, the chronic outward force provided by the stent device 100 following implantation in a blood vessel of the cerebral dural venous sinuses is sufficient to eliminate or significantly reduce the pulsatile tinnitus experienced by a patient. The chronic outward force produced by the stent device 100 can be measured using a radial force testing equipment.

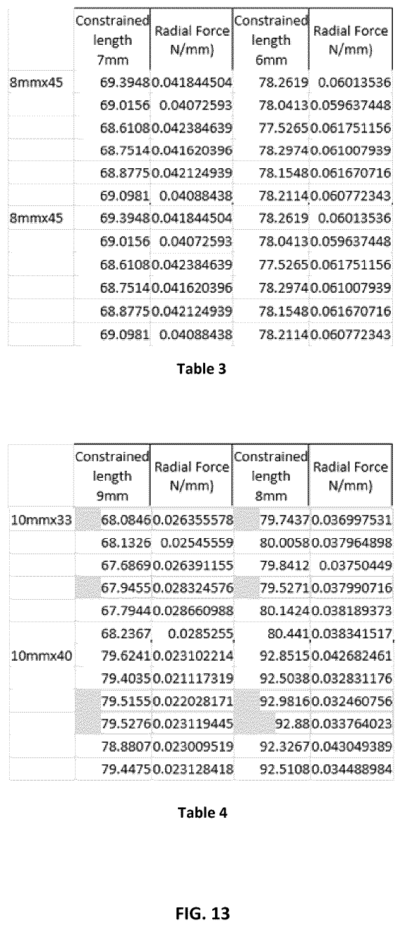

The resistive radial force of the stent device 100 is the force generated by the stent to resist compression (e.g., compression applied by the blood vessel in which the stent device 100 is implanted). The resistive radial force of the stent device 100 can be optimized to ensure that the stent device 100 remains patent following implantation without the stent device 100 applying excess pressure on the walls of the blood vessel within which the stent device 100 is implanted. In some implementations, the resistive radial force of the stent device 100 is in a range of 0.170 N/mm and 0.700 N/mm. In some implementations, the resistive radial force of the stent device 100 is in a range of 0.300 N/mm and 0.500 N/mm. In some implementations, the resistive radial force of the stent device 100 is in a range of 0.300 N/mm and 0.400 N/mm. In some implementations, the resistive radial force of the stent device 100 is in a range of 0.1651 N/mm and 0.2998 N/mm. In some implementations, the resistive radial force of the stent device 100 is in a range of 0.20299 N/mm to 0.54337 N/mm Table 3 in FIG. 13 depicts the resistive radial force measured for two example stent devices 100 having a diameter of 8 mm and Table 4 in FIG. 14 depicts the resistive radial force measured for two example stent devices 100 having a diameter of 10 mm.

As can be seen in FIGS. 1 and 2, the stent device 100 includes a flared crown 110, 112 at each of the proximal end 106 and the distal end 108 of the stent device 100. The flared crowns 110, 112 allow for securement of the stent device 100 to the blood vessel within which the stent device 100 is deployed. For example, when stent devices are deployed in veins, it can be difficult to maintain the position of the stent device within the vein because veins do not have elastic lamina or muscular tissue similar to that present in arteries. The crowns 110, 112 of the stent device 100 help maintain the position of the stent device 100 within a blood vessel once deployed, which enables the stent device 100 to be deployed and maintain its positioning within a vein, such as within the cerebral dural venous sinuses of a patient. In some implementations, the unconstrained diameter of each of the crowns 110, 112 is in a range of 1 mm to 2 mm larger than the unconstrained diameter 126 of the stent device body 104. For example, in some implementations, the unconstrained diameter of each of the crowns 110, 112 is in a range of 7 mm to 12 mm.

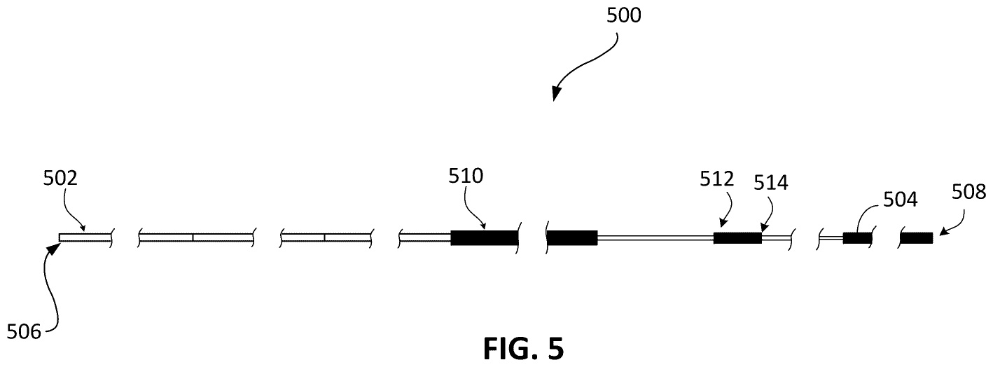

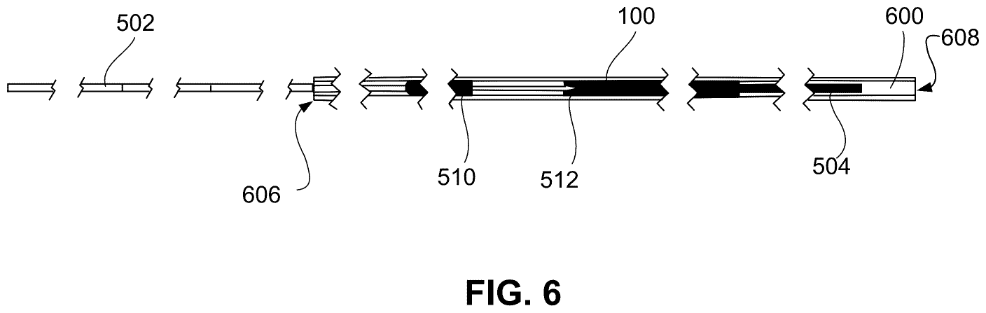

Referring to FIGS. 5 and 6, the stent device 100 is configured to be implanted in the cerebral dural venous sinuses of a patient using an insertion device 500 and a catheter 600. As can be seen in FIG. 5, the insertion device 500 includes a pusher rod 502, a stent body coil 504 positioned at a distal end 508 of the pusher rod 502, a radiopaque pusher body coil 510 positioned over a portion of the pusher rod 502, and a polymer tube 512 positioned over the pusher rod 502 between the stent body coil 504 and the radiopaque pusher body coil 510.

In some implementations, the catheter 600 is a 5 French guide catheter. In some implementations, the catheter 600 is a guide catheter that is smaller than 5 French. In some implementations, the inner diameter of the catheter 600 is 0.058 inches and the outer diameter of the catheter is 0.066 inches. In some implementations, the length of the catheter is in a range of 100 centimeters and 150 centimeters. In some implementations, the length of the catheter 600 is 105 centimeters. In some implementations, the length of the catheter 600 is 115 centimeters. In some implementations, the length of the catheter 600 used to implant the stent device 100 is selected based on the anatomy of the patient. As will be described in further detail herein, the catheter 600 can be inserted into and guided through one or more blood vessels of dural venous sinuses of the patient to a target vessel before inserting the stent device 100 into the catheter 600.

In some implementations, the pusher rod 502 tapers between the proximal end 506 of the pusher rod 502 and the distal end 508 of the pusher rod 502. The pusher rod 502 can be formed of any suitable material including, but not limited to, stainless steel (e.g., 304V stainless steel), cobalt-chromium, or nitinol (NiTi). In some implementations, the length of the pusher rod 502 is in a range of 196 centimeters to 201 centimeters. In some implementations, the diameter of the pusher rod 502 is in a range of 0.025 inches to 0.040 inches. In some implementations, the diameter of the pusher rod 502 is 0.028 inches. As will be described in further detail herein, the stent device 100 can be placed over the pusher rod 502 and the pusher rod 502 and stent device 100 can be inserted together through the catheter 600 to deliver the stent device 100 to a target location for implantation.

Referring to FIG. 5, the stent body coil 504 of the insertion device 500 is attached to the proximal end the of pusher rod 502. The stent body coil 504 is formed of a coil of metal wire and is sufficiently flexible to prevent injury to the blood vessel of the patient as the insertion device 500 is advance through the patient's vasculature during implantation of the stent device 100. Referring to FIG. 6, the stent body coil 504 is configured to extend through about half of the length 124 of the stent device 100 when the stent device 100 is loaded onto the insertion device 500 for implantation. The stent body coil 504 provides column strength and kink resistance to the pusher rod 502 and the stent device 100 as the pusher rod 502 and the stent device 100 are advanced through the catheter 600 during delivery of the stent device 100 to a target vessel. In some implementations, the length of the stent body coil 504 is in a range of 8 centimeters to 12 centimeters. In some implementations, the diameter of the stent body coil 504 is in a range of 0.015 inches to 0.035 inches. In some implementations, the diameter of the stent body coil 504 is 0.023 inches.

The pusher body coil 510 is attached to the pusher rod 502 between the proximal end 506 of the pusher rod 502 and the polymer tube 512. In some implementations, the distance between the distal end 508 of the pusher rod 502 and a distal end of the pusher body coil 510 is in a range of 12 centimeters to 16 centimeters. The pusher body coil 510 is formed of a coil of metal wire that is sufficiently flexible to prevent kinking of the pusher rod 502 as the pusher rod 502 is advanced through the patient's vasculature during implantation of the stent device 100. In some implementations, the pusher body coil is formed of stainless steel (e.g., 304V stainless steel), nitinol (NiTi), a platinum-tungsten alloy, platinum, or a combination thereof. In some implementations, the pusher body coil 510 is formed of a radiopaque material, such as platinum. As a result, the pusher body coil 510 medical imaging can be used during an implantation procedure in order to track the position of the insertion device 500 within the patient's vasculature throughout the implantation procedure. As a result, the clinician can monitor the position of the pusher body coil 510 within the patient's blood vessel in real time to ensure accurate placement of the stent device 100.

In some implementations, the length of the pusher body coil 510 is in a range of 20 centimeters to 40 centimeters. In some implementations, the length of the pusher body coil 510 is 23 centimeters. In some implementations, the diameter of the pusher body coil 510 is in a range of 0.035 inches to 0.060 inches. In some implementations, the diameter of the pusher body coil 510 is 0.056 inches.

Still referring to FIG. 6, the polymer tube 512 is attached to the pusher rod 502 along the length of the pusher rod 502 between the stent body coil 504 and the pusher body coil 510. In some implementations, the distance between the distal end 508 of the pusher rod 502 and a distal end of the polymer tube 512 is in a range of 11 centimeters to 15 centimeters. The polymer tube 512 can be formed of any suitable polymer material, including, but not limited to polyimide, high density polyethylene (HDPE), fluorinated ethylene propylene (FEP), or nylon.

In some implementations, a platinum tungsten (PtW) coil 514 is coupled to the pusher rod 502 underneath the polymer tube 512. As a result of the presence of the PtW coil 514 underneath the polymer tube 512, the position of the polymer tube 512 can be tracked throughout an implantation procedure using medical imaging. As will be described in further detail herein, the polymer tube 512 together with the catheter 600 are configured to releasably couple the proximal end 106 of the stent device 100 to the pusher rod 502 during implantation of the stent device 100. Therefore, by tracking the position of the polymer tube 512 during the implantation procedure, the position of the proximal end 106 of the stent device 100 can be determined in real-time throughout the implantation procedure.

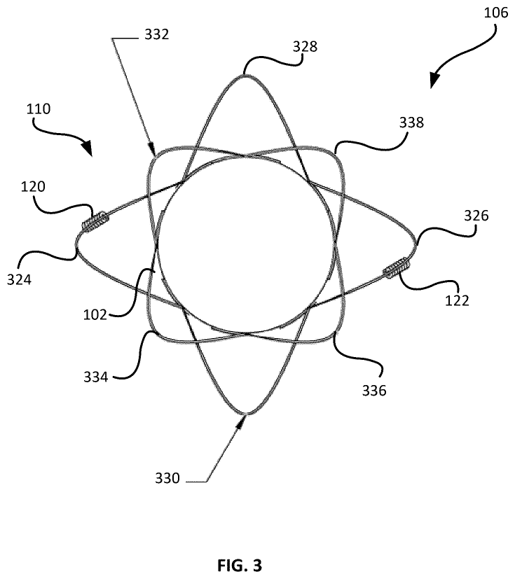

Referring to FIGS. 2, 3, and 7, the stent device 100 includes two marking coils 120, 122 positioned on the proximal end 106 of the stent body 104 and configured to interact with the polymer tube 512 and the catheter 600 to maintain the position of the stent device 100 along the pusher rod 502 prior to implantation of the stent device 100 in a blood vessel. As can be seen in FIGS. 2 and 3, the marking coils 120, 122 are each formed of a length of wire that is coiled around opposite loops 324, 326 of the stent body 104 at the proximal end 106 of the stent body 104. As can be seen in FIG. 3, the proximal end 106 of the stent body 104 includes four long loops 324, 326, 328, 330 and four short loops 332, 334, 336, 338, and the marking coils 120, 122 are coupled to the stent body 104 along two of long loops 324, 326 opposite each other. In some implementations, the marking coils 120, 122 are soldered to the stent body 104. In some implementations, the marking coils 120, 122 are glued to the stent body 104 using UV-curable adhesives or epoxy. In some implementations, the marking coils 120, 122 are formed of radiopaque wire, which allows the marking coils 120, 122 to be visualized using medical imaging in real-time during implantation of the stent device 100. Therefore, the position of the proximal end 106 of the stent device 100 can be determined by tracking the position of the marking coils 120, 122 using medical imaging during the implantation procedure. In some implementations, the marking coils 120, 122 are formed of DFT wire. For example, the marking coils 120, 122 can be formed of a nickel-titanium alloy (e.g., nitinol) tube that is filled with platinum, a platinum-tungsten alloy, gold, barium sulfate, or a combination thereof.

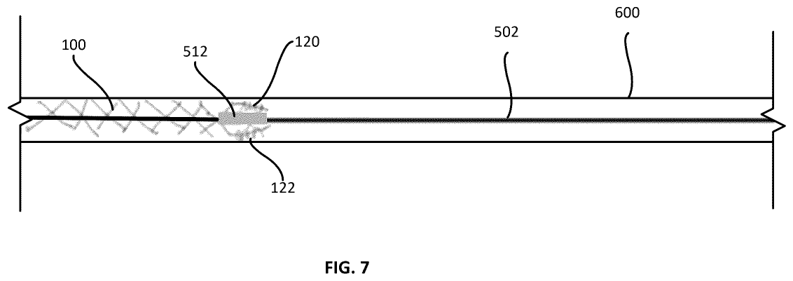

As depicted in FIG. 7, when the stent device 100 is loaded onto the insertion device 500 for implantation of the stent device 100, the marking coils 120, 122 are positioned over and frictionally engage with the polymer tube 512. During implantation of the stent device 100, the insertion device 500 with the stent device 100 releasably coupled thereto is inserted into and passed through the catheter 600. As can be seen in FIG. 7, as the insertion device 500 and stent device 100 are passed through the catheter 600, the marking coils 120, 122 are in contact with and positioned between the polymer tube 512 and an inner surface of the catheter 600. In addition, as depicted in FIG. 7, the marking coils 120, 122 extend proximally beyond the polymer tube 512 and engage with a proximal surface of the polymer tube 512. The friction generated by the contact between the marking coils 120, 122 and the polymer tube 512 and the catheter 600 prevents movement of the stent device 100 within the catheter 600 while the catheter 600 is positioned over the marking coils 120, 122 and polymer tube 512. Once the distal end 608 of the catheter 600 is properly positioned within a blood vessel for delivery of the stent device 100, the catheter 600 is withdrawn proximally while the position of the pusher rod 502 is maintained. Once the catheter 600 is withdrawn proximally over the polymer tube 512 such that the polymer tube 512 and the marking coils 120, 122 are positioned outside the catheter 600, the proximal end 106 of the stent body 104 expands, which disengages the marking coils 120, 122 from the polymer tube 512. As a result, the stent device 100 is released from the insertion device 500 and is implanted within the blood vessel.

A method of implanting the stent device 100 into a blood vessel will now be described in reference to FIGS. 2, 4-7, 9, and 10.

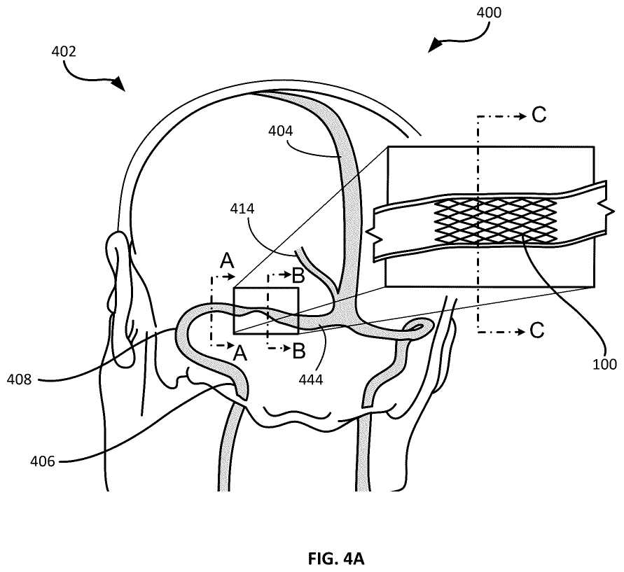

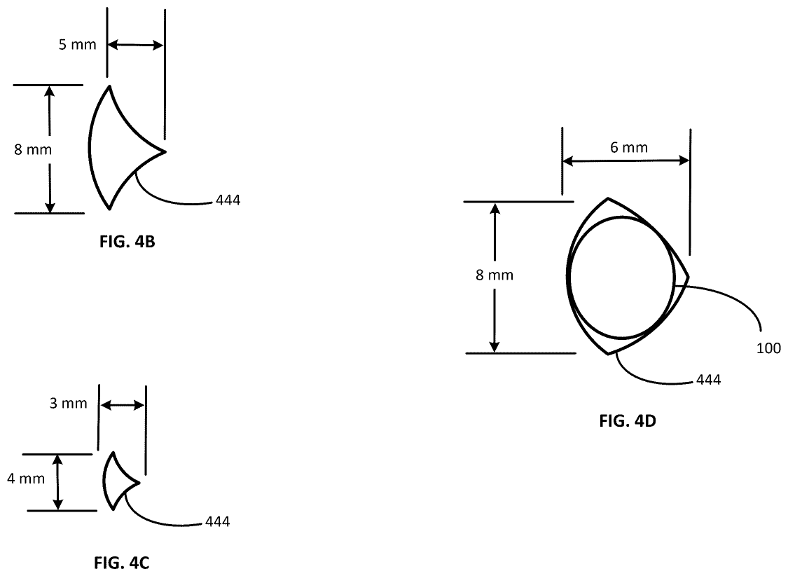

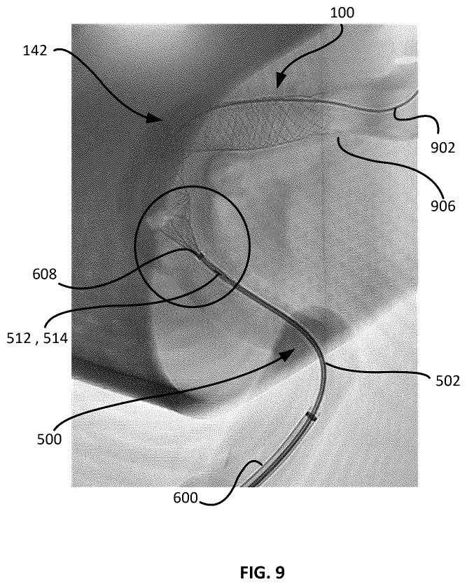

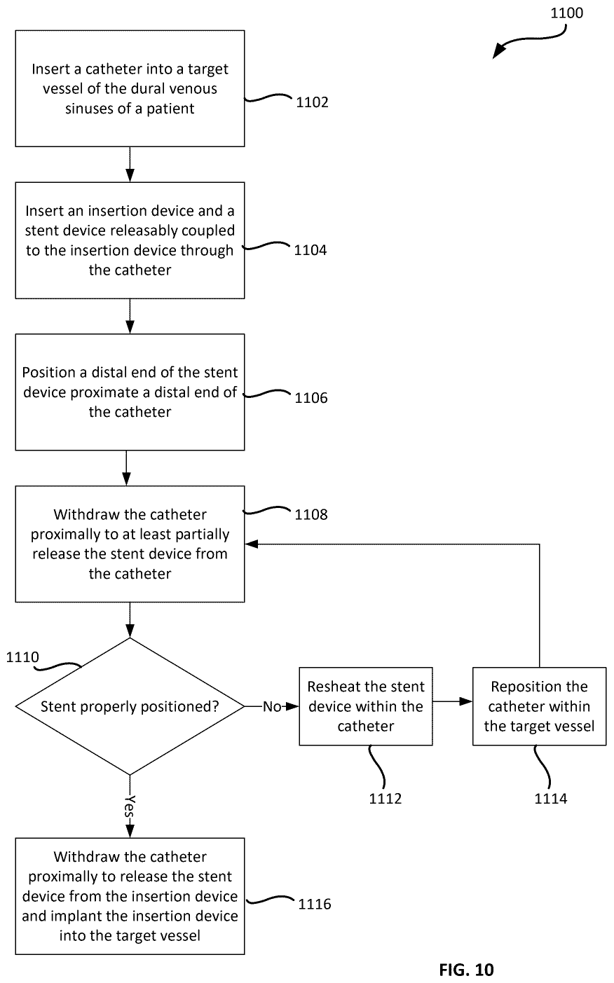

Referring to FIG. 10, a method of implanting a stent device (e.g., stent device 100 of FIGS. 1-9) includes inserting a catheter (e.g., catheter 600 of FIG. 6) into a target vessel of the cerebral dural venous sinuses 400 of a patient (1102). As depicted in FIGS. 4A and 4B, the portions of the transverse sinus 444 that are not experiencing stenosis have a lumen that is generally 8 mm in height and 5 mm in width. In comparison, the portions of the transverse sinus that are not experiencing stenosis have a reduced lumen, for example, that is generally 4 mm in height and 3 mm in width, as depicted in FIGS. 4A and 4C. In order to treat the stenosis of the transverse sinus 444, the stent device 100 can be implanted within the transverse sinus 444 of the patient, as depicted in FIG. 4A. In order to implant the stent device 100 within the transverse sinus 444 of the patient, a 5 French diameter catheter 600 is passed through the jugular bulb 406 and the sigmoid sinus 408 and into the transverse sinus 444. Referring to FIG. 9, in some implementations, inserting the catheter 600 into a target vessel 906 is performed by accessing the femoral vein of the patient 402 using a micro-puncture kit, placing a short sheath in the femoral vein of the patient 402, guiding the catheter 600 through the short sheath and into the femoral vein using a dilator and guidewire 902. Once inserted into the femoral vein, the guidewire 902 and catheter 600 can be tracked using medical imaging (e.g., fluoroscopy) and advanced through the body to the target vessel 906 (e.g., transverse sinus 444). In some implementations, the catheter 600 is inserted into the target blood vessel 906 without the insertion device 500 or the stent device 100. In other words, the catheter 600 can be placed in the target blood vessel 906 prior to inserting the insertion device 500 or the stent device 100 into the catheter 600.

Once the catheter is inserted into the target vessel, an insertion device and a stent device releasably coupled to the insertion device are inserted into the catheter (1104), for example, as depicted in FIG. 6. Referring to FIG. 5, the stent device 100 is coupled to the insertion device 500 by inserting the pusher rod 502 through the lumen of the stent device 100 until the marking coils 120, 122 at the proximal end 106 of the stent device 100 are in contact with and positioned at least partially over the polymer tube 512. Once the stent device 100 is coupled to the pusher rod 502 with the marking coils 120, 122 positioned over the polymer tube 512, the pusher rod 502 is inserted into the proximal end 606 of the catheter 600.

Once inserted into the catheter, the insertion device together with the stent device is advanced distally through the catheter until the distal end of the stent device is positioned proximate the distal end of the catheter (1106). As the pusher rod 502 is advanced distally through the catheter 600, the friction generated by the contact of the marking coils 120, 122 with the inner surface of the catheter 600 and with the polymer tube 512 is sufficient to prevent the stent device 100 from moving within the catheter 600 relative to the pusher rod 502 while the stent device 100 is advanced through the catheter 600. As a result, the stent device 100 can be advanced and retracted within the catheter 600 via movement of the pusher rod 502 so long as the marking coils 120, 122 remain in contact with the inner surface of the catheter 600 and with the polymer tube 512.

Once the distal end of the catheter is properly positioned within the target vessel and the distal end of the stent device is positioned proximate the distal end of the catheter, the catheter is withdrawn proximally to at least partially release the stent device from the catheter (1108). FIG. 9 depicts an example of real-time medical imaging of the stent device 100 during implantation of the stent device 100. As can be seen in FIG. 9, as the catheter 600 is withdrawn proximally, the portion 142 of the stent device 100 that is no longer contained inside the catheter 600 self-expands to apply a force to the target vessel 906. As previously discussed, the stent device 100 is formed of radiopaque DFT wire 102 and, as a result, the position of the expanded stent device 100 can be monitored in real-time during the implantation procedure, as depicted in FIG. 9. In addition, the PtW coil 514 underneath the polymer tube 512 502 is radiopaque, and since the proximal end 106 of the stent device 100 is coupled to the polymer tube 512 during insertion via marking coils 120, 122, the position of the proximal end 106 of the stent device 100 can be tracked during implantation by monitoring the position of the polymer tube 512. In addition, in some implementation, the marking coils 120, 122 are formed of a radiopaque material, such as radiopaque DFT wire, and the proximal end 106 of the stent device 100 can be tracked during implantation by monitoring the position of the marking coils 120, 122 using medical imaging. By monitoring the position of the polymer tube 512 and/or the proximal end 106 of the stent device 100 within the catheter 600, a clinician can determine how close the distal end 608 of the catheter 600 is from the proximal end 106 of the stent device 100, which relates to how close the stent device 100 is from being fully deployed from the catheter 600 and, as a result, no longer resheathable into the catheter 600.

Prior to fully deploying the stent device from the catheter, a clinician uses medical imaging to visualize the position of the stent device within the target vessel to determine whether the stent device is properly positioned within the target vessel (1110). If the clinician determines that the stent device 100 is not properly positioned within the target vessel 906, the clinician can advance the catheter 600 distally to resheath the stent device 100 within the catheter 600 (1112) and the catheter 600 can be repositioned within the target vessel 906 (1114). Once the catheter 600 is repositioned within the target vessel 906, the process of partially releasing the stent device 100 from the catheter 600 and checking the positioning of the stent device 100 can be repeated.

In addition, due to the self-expanding material used to form the stent device 100 (e.g., Nitinol), a clinician can use the catheter 600 to apply forward pressure on the portion of the stent device 100 that has been deployed from the catheter 600, without resheathing the device 100. By applying forward (e.g., distal) pressure on the deployed portion of the stent device 100, the stent device 100 can be positioned to more closely appose the walls of the target blood vessel 906 and better contour to the target blood vessel 906. In addition, by applying forward pressure on the stent device 100 using the catheter 600 while the stent device 100 is partially deployed, the stent device 100 can be used to further open a stenosis of the target blood vessel 905 during deployment of the stent device 100 within the blood vessel 906.

As previously discussed, the friction generated by the contact of the marking coils 120, 122 with the inner surface of the catheter 600 and the polymer tube 512 is sufficient to prevent the stent device 100 from moving relative to the pusher rod 502 while being advanced through the catheter 600. As a result, the stent device 100 can be resheathed into the catheter 600 so long as the marking coils 120, 122 and the polymer tube 512 are contained within the catheter 600. In some implementations, the stent device 100 can resheathed within the catheter 600 with up to 90% of the length of the stent body 104 deployed from the catheter 600. Thus, the stent device 100 can be resheathed within the catheter 600 when 90% or less of the stent device 100 has been deployed from the catheter 600. The ability to resheath the stent device 100 into the catheter 600 provides several advantages, including enabling the clinician to reposition the stent device 100 when the initial deployment location was improperly selected, the stent device 100 slips distally or proximally during deployment, or if the diameter or length of the selected stent device 100 is determined to be improper based on the patient's anatomy.

Once the clinician has confirmed that the stent device is properly positioned within the target vessel, the clinician withdraws the catheter proximally to release the stent device from the insertion device and implant the stent device within the target vessel (1116). As previously discussed, the friction generated by the contact of the marking coils 120, 122 with the inner surface of the catheter 600 and the polymer tube 512 is sufficient to prevent the stent device 100 from moving relative to the pusher rod 502. In order to release the stent device 100 from the insertion device 500, the catheter 600 is withdrawn proximally while maintaining the position of the pusher rod 502 until the marking coils 120, 122 and polymer tube 512 are deployed out of the distal end 608 of the catheter 600 and positioned outside of the catheter 600. Once the proximal end 106 of the stent device 100 is outside of the catheter 600, the proximal end 106 of the stent device 100 expands such that the marking coils 120, 122 are no longer frictionally engaged with the polymer tube 512. Thus, deploying the marking coils 120, 122 and polymer tube 512 outside the catheter 600 releases the stent device 100 from the insertion device 500 and implants the stent device 100 inside the target vessel 906. In some implementations, the force required to deploy the stent device 100 outside the catheter 600 is in a range of 0.125 lbs. of force to 2 lbs. of force.

Once the stent device 100 is deployed the catheter 600, the stent device 100 self-expands to an expanded state within the target vessel 906 in the patient's dural venous sinuses. The expansion of the stent device 100 is in the expanded state, the stent device 100 contacts the inner walls of the target vessel 906, as depicted in FIG. 4D, and applies to the blood vessel 906 that is sufficient to change the blood flow within the blood vessel 906 from pulsatile flow or turbulent flow to laminar flow. For example, as depicted in FIG. 4D, once implanted, the stent device 100 contacts and applies a chronic outer force to the inner wall of the transverse sinus to cause the lumen of the transverse sinus to increase to allow for increased blood flow through the transverse sinus 444. In some implementations, following implantation of the stent device 100, the lumen of the transverse sinus 444 is expanded to have a height of 8 mm and a width of 6 mm. As a result, the chronic outward force provided by the stent device 100 following implantation in the target vessel 906 of the cerebral dural venous sinuses is sufficient to eliminate or significantly reduce the pulsatile tinnitus experienced by a patient. Once the stent device 100 is fully deployed from the catheter 600 and expanded within the lumen of the target blood vessel 906 (i.e., once the stent device 100 is implanted in the blood vessel), the position of the stent device 100 the contact between the stent device 100 and the inner walls of the blood vessel, and in particular the contact between the flared crowns 110, 112 of the stent device 100 and the blood vessel, permanently fixes the position of the stent device 100 within the blood vessel. Therefore, the stent device 100 cannot be resheathed or removed from the blood vessel 906 once the stent device 100 has been fully deployed from the catheter 600 and implanted within the blood vessel.

Once the stent device 100 is released from the insertion device 500, the insertion device 500 can be withdrawn proximally through the catheter 600 and removed from the patient. Once the stent device 100 is implanted in the target vessel 906 and the insertion device 500 has been removed, the catheter 600 can be further withdrawn proximally and removed from the patient.

While certain embodiments have been described, other embodiments are possible.

For example, while the stent device 100 has been described as having flared crowns 110, 112 at both the proximal and distal ends 106, 108 of the stent body 104, in some implementations, the stent device only includes a flared crown on the one end of the stent body. For example, in some implementations, the stent device includes a flared crown only on the proximal end 106 of the stent body 104. In some implementations, the stent device includes a flared crown only on the distal end 108 of the stent body 104. In some implementations, the stent device does not include flared crowns, but rather has a substantially constant diameter along the entire length 124 of the stent body 104.

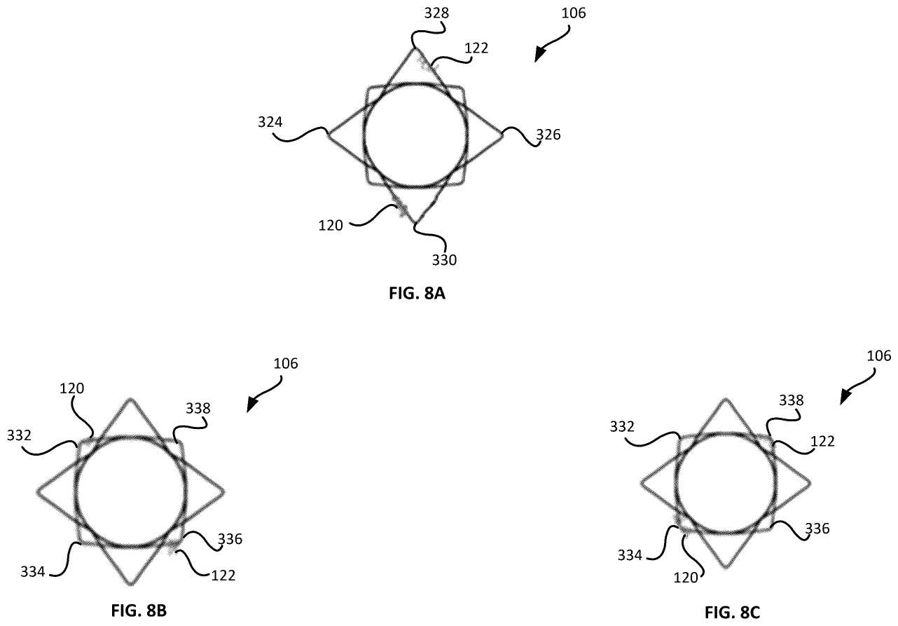

In addition, while the marking coils 120, 122 have been depicted as being coupled to long loops 324, 326 of the stent device 100, the marking coils 120, 122 can be coupled to other opposing loops of proximal end 106 of the stent device 100. For example, as depicted in FIG. 8A, the marking coils 120, 122 can be coupled to another set of long loops 328, 330 of the stent device 100. In some implementations, the marking coils 120, 122 are coupled opposing short loops of the proximal end 106 of the stent device 100. For example, the marking coils 120, 122 can be coupled a first pair of opposing short loops 332, 336, as depicted in FIG. 8B, or a second pair of opposing short loops 334, 338, as depicted in FIG. 8C.

Further, while the stent device 100 has been described as being releasably coupled to insertion device 500 using marking coils 120, 122, in some implementations, the stent device does not include marking coils, and the pusher body coil 510 is covered in PET heat shrink that generates friction between the stent device 100 and the pusher body coil 510 when the stent device 100 is positioned over the pusher body coil 510. The friction generated by the PET heat shrink prevents movement of the stent device 100 moving relative to the pusher rod 502 while the stent device 100 is advanced through the catheter 600.

In addition, while the stent device 100 has been described as having a braided stent body 104 formed by braiding a single wire 102, in some implementations, the stent body is formed of a single piece of material that is laser cut to form a series of cells. In some implementations, the stent device is an open celled laser cut stent in which at least one edge of each of the cells in the stent device are not connected to the edges of the adjacent cells. In some implementations, the stent device is a closed celled laser cut stent in which all of the edges of each cell in the stent device are not connected to the edges of the adjacent cells.

While the stent device 100 has been depicted as being deployed in the transverse sinus 444 of the patient, the stent device 100 can be deployed and implanted in other blood vessels of a patient 402. For example, the stent device 100 can be implanted within and extend through one or more blood vessels of the cerebral dural venous sinuses 400 including, but not limited to, the superior sagittal sinus 404, the jugular bulb 406, the sigmoid sinus 408, the straight sinus 414, and the transverse sinus 444. In some implementations, the stent device 100 is implanted within and extends across the sigmoid sinus 408 and the transverse sinus 444. For example, the stent device 100 can be deployed within the cerebral venous sinuses such that the stent device 100 extends between the torcula of the cerebral venous sinuses, which is the origin of the transverse sinus 444, to the end of the sigmoid sinus 408 above the jugular bulb. In some implementations, the stent device 100 is implanted within and extends across the jugular bulb 406, the sigmoid sinus 408, and the transverse sinus 444.

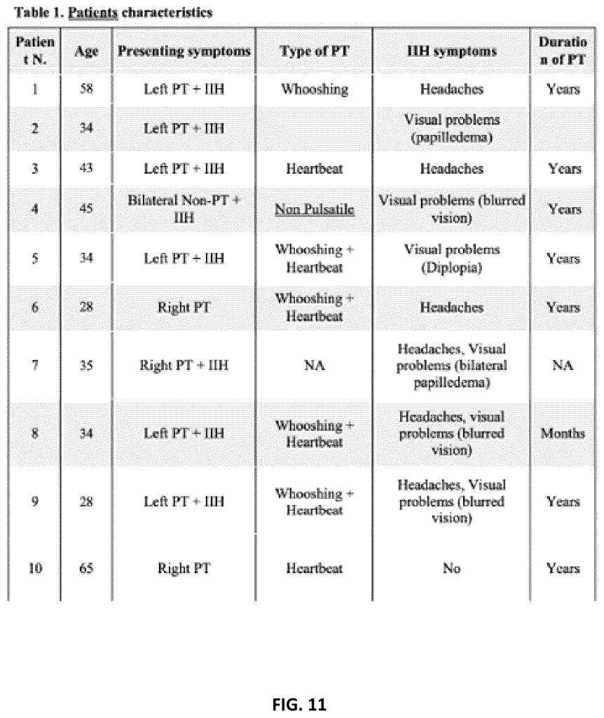

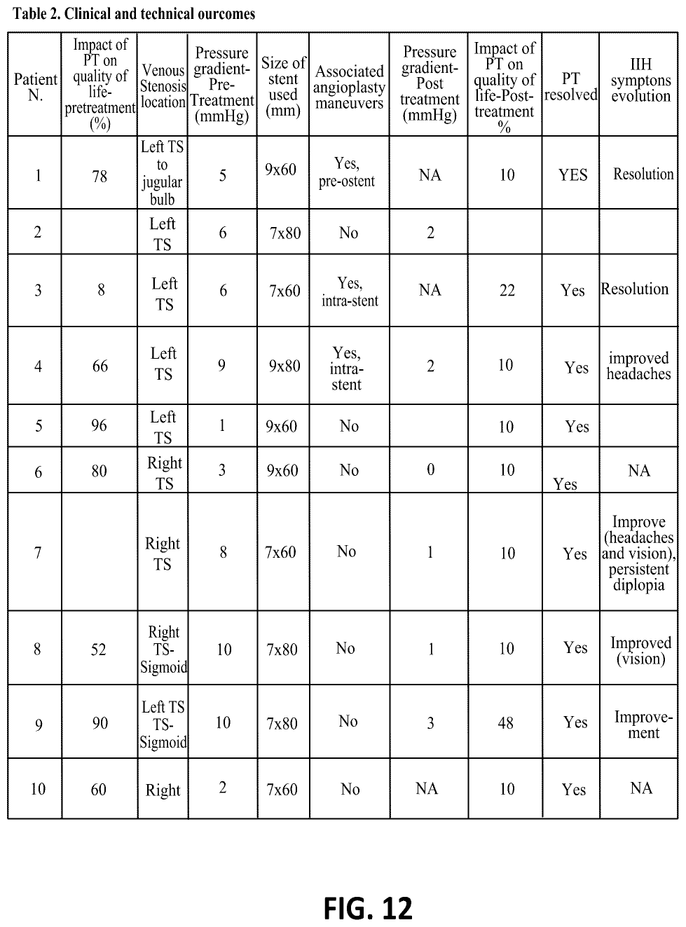

A clinical study was conducted to treat patients experiencing pulsatile tinnitus using the stent device 100 described herein. The pulsatile tinnitus symptoms presented by each patient during the study is summarized in Table 1 depicted in FIG. 11. During the study, the stent device 100 was implanted across one or more blood vessels of the cerebral venous sinuses of the respective patient. Table 2 depicted in FIG. 12 summarizes the clinical outcomes of the stent implantation for each patient in the study. As can be seen in FIG. 12, each of the patients treated with the stent device 100 during the study experienced a reduction in pressure gradient across the cerebral venous sinuses and the pulsatile tinnitus for each patient in the study was resolved following implantation of the stent device 100. In addition, each of the patients in the study experienced an improvement or complete resolution of idiopathic intracranial hypertension symptoms following implantation of the stent device 100. Thus, the clinical study summarized in Tables 1 and 2 confirmed the ability of the stent device 100 to treat pulsatile tinnitus.

A number of embodiments have been described. Nevertheless, it will be understood that various modifications may be made without departing from the spirit and scope of the disclosure. Accordingly, other embodiments are within the scope of the following claims.

1. A stent device comprising:
a single wire forming a braided body that extends between a proximal end and a distal end, wherein:
the single wire is formed of drawn filled tube (DFT);
the braided body has a braid density in a range of 15 programmable picks per inch (PPI) to 21 PPI;
the single wire forms a flared crown on at least one of the proximal end or the distal end of the braided body;
the flared crown has an unconstrained diameter that is 1 mm to 2 mm larger than an unconstrained diameter of the braided body for maintaining a position of the stent device within a vein once the stent device is deployed; and
the stent device is configured for implantation in a cerebral dural venous sinus and, based at least in part on the unconstrained diameter of the braided body and the braid density of 15 PPI to 21 PPI, is configured to generate a chronic radially outward force sufficient to change blood flow within the cerebral dural venous sinus from pulsatile flow or turbulent flow to laminar flow and to generate a resistive radial force in a range of 0.3 N/mm to 0.5 N/mm when implanted.

2. (canceled)

3. The stent device of claim 1, wherein the chronic radially outward force generated by the stent device is at least 0.036 N/mm.

4. The stent device of claim 1, wherein the chronic radially outward force generated by the stent device is in a range of at 0.0197 N/mm and 0.0433 N/mm.

5. The stent device of claim 1, wherein an unconstrained length of the braided body is 45 mm.

6. The stent device of claim 5, wherein a diameter of the wire is between 0.0035 inches and 0.0065 inches.

7. (canceled)

8. The stent device of claim 1, wherein an unconstrained length of the braided body is 60 mm.

9. The stent device of claim 8, wherein a diameter of the wire is between 0.0035 inches and 0.0065 inches.

10. (canceled)

11. The stent device of claim 1, further comprising at least one coil of DFT wire coiled about the braided body at the proximal end, wherein the at least one coil is configured to releasably couple the stent device to an insertion device.

12. (canceled)

13. A system comprising:
a stent device comprising a single wire forming a braided body that extends between a proximal end and a distal end, wherein:
the single wire is formed of drawn filled tube (DFT);
the braided body has a braid density in a range of 15 programmable picks per inch (PPI) to 21 PPI;
the single wire forms a flared crown on at least one of the proximal end or the distal end of the braided body;
the flared crown has an unconstrained diameter that is 1 mm to 2 mm larger than an unconstrained diameter of the braided body for maintaining a position of the stent device within a vein once the stent device is deployed; and
the stent device configured for implantation in a cerebral dural venous sinus and, based at least in part on the unconstrained diameter of the braided body and the braid density of 15 PPI to 21 PPI, is configured to generate a chronic radially outward force sufficient to change blood flow within the cerebral dural venous sinus from pulsatile flow or turbulent flow to laminar flow and to generate a resistive radial force in a range of 0.3 N/mm to 0.5 N/mm when implanted;
an insertion device comprising:
a pusher rod;
a stent body coil positioned proximate an end of the pusher rod, wherein the stent body coil is configured to extend through at least a portion of the stent device when the stent device is coupled to the insertion device;
a radiopaque pusher body coil coupled to a portion of the pusher rod;
a polymer tube coupled to the pusher rod between the stent body coil and the pusher body coil; and
a catheter configured to enclose the stent device and the pusher rod.

14. The system of claim 13, wherein:
stent device further comprises at least one coil of DFT wire coiled about the braided body at the proximal end, wherein the at least one coil is configured to releasably couple the stent device to an insertion device; and
the stent device is releasably coupled to the insertion device by friction between the at least one coil, the polymer tube, and an inner surface of the catheter.

15. The system of claim 14, wherein, when the stent device is coupled to the insertion device, the at least one coil is positioned proximal to the polymer tube.

16. The system of claim 14, wherein the at least one coil comprises two coils positioned on opposite loops of the proximal end of the braid body.

17. (canceled)

18. (canceled)

19. The system of claim 13, wherein the stent device is resheathable into the catheter when approximately 90% or less of the braid body has been deployed outside the catheter.

20. The system of claim 13, wherein the catheter is a 5 French catheter.

21. The system of claim 13, wherein the pusher body coil is configured to prevent kinking of the pusher rod during insertion of the stent device in the cerebral dural venous sinus.

22. A method comprising:
inserting a catheter into a target vessel of dural venous sinuses of a patient;
inserting an insertion device and a stent device releasably coupled to the insertion device through the catheter;
positioning a distal end of the stent device proximate a distal end of the catheter; and
withdrawing the catheter proximally to release the stent device from the insertion device and implant the insertion device into the target vessel,
wherein:
the stent device comprises a single wire forming a braided body that extends between a proximal end and a distal end;
the single wire is formed of drawn filled tube (DFT);
the braided body has a braid density in a range of 15 programmable picks per inch (PPI) to 21 PPI;
the single wire forms a flared crown on at least one of the proximal end or the distal end of the braided body;
the flared crown has an unconstrained diameter that is 1 mm to 2 mm larger than an unconstrained diameter of the braided body for maintaining a position of the stent device within a vein once the stent device is deployed; and
the stent device is configured for implantation in a cerebral dural venous sinus and, based at least in part on the unconstrained diameter of the braided body and the braid density of 15 PPI to 21 PPI, the stent device is configured to generate a chronic outward force sufficient to change blood flow within the cerebral dural venous sinus from pulsatile flow or turbulent flow to laminar flow and to generate a resistive radial force in a range of 0.3 N/mm to 0.5 N/mm when implanted.

23. (canceled)

24. The method of claim 22, wherein:
stent device further comprises at least one coil of DFT wire coiled about the braided body at the proximal end;
the insertion device comprises a pusher rod and a polymer tube positioned over the pusher rod; and
when the stent device is coupled to the insertion device, the at least one coil is positioned proximal to the polymer tube.

25. The method of claim 24, wherein releasing the stent device from the insertion device comprises withdrawing the catheter proximally until the polymer tube is deployed outside a distal end of the catheter.

26. The method of claim 22, wherein the catheter is a 5 French catheter.

27. The method of claim 22, further comprising, prior to releasing the stent device from the insertion device:
resheathing the stent device within the catheter; and
repositioning the catheter within the target vessel.

28. The method of claim 27, wherein resheathing of the stent device into the catheter is performed when approximately 90% or less of the stent device has been deployed outside the catheter.

29. The method of claim 22, further comprising withdrawing the insertion device proximally out of the catheter and withdrawing the catheter out of the dural venous sinuses of the patient.

30. The method of claim 22, wherein implantation of the stent device in the target vessel is performed using medical imaging.

31. The stent device of claim 11, wherein the insertion device comprises:
a pusher rod;
a stent body coil positioned proximate an end of the pusher rod, wherein the stent body coil is configured to extend through at least a portion of the stent device when the stent device is coupled to the insertion device;
a radiopaque pusher body coil coupled to a portion of the pusher rod;
a polymer tube coupled to the pusher rod between the stent body coil and the pusher body coil; and
a catheter configured to enclose the stent device and the pusher rod.

32. The stent device of claim 31, wherein the stent device is releasably coupled to the insertion device by friction between the at least one coil, the polymer tube, and an inner surface of the catheter.

33. The stent device of claim 31, wherein the stent device is configured to be resheathable into the catheter when approximately 90% or less of the braided body has been deployed outside the catheter.
